(19) United States
(12) Patent Application Publication
(10) Pub. No.: US 2023/0193178 A1
(43) Pub. Date: Jun. 22, 2023

(54) METHODS AND SYSTEMS TO PRINT AND MATURE TISSUES OVER TIME IN A THREE-DIMENSIONAL SUPPORT MATRIX

(71) Applicant: The General Hospital Corporation, Boston, MA (US)

(72) Inventors: Harald C. Ott, Wenham, MA (US); Joshua Gershlak, Somerville, MA (US)

(21) Appl. No.: 17/908,810

(22) PCT Filed: Mar. 5, 2021
(86) PCT No.: PCT/US2021/021162

§ 371 Date: Sep. 1, 2022

Related U.S. Application Data
(60) Provisional application No. 62/985,408, filed on Mar. 5, 2020.

(51) Int. Cl. C12M 3/00; C12M 1/12; C12M 1/26; C12M 1/34; C12N 5/00; B33Y 10/00; B33Y 70/00; B33Y 80/00

(52) U.S. Cl. CPC C12M 21/08; C12M 25/14; C12M 33/04; C12M 41/12; C12N 5/0062; B33Y 10/00; B33Y 70/00; B33Y 80/00; C12N 2539/10; C12N 2533/70; C12N 2537/10

(57) ABSTRACT

A method of forming a tissue or an organ, including: disposing, in a support medium in a gel state, a composition comprising a live biologic; changing a state of the support medium from the gel state to a solid state; and supporting, in the support medium at the solid state, the live biologic in the composition.

### CROSS REFERENCE TO RELATED APPLICATIONS

This application claims the benefit of U.S. Provisional Application No. 62/985,408 filed Mar. 5, 2020, the contents of which are incorporated herein by reference in their entireties.

### TECHNICAL FIELD

The present invention relates generally to tissue bioprinting, and more specifically, to three-dimensional tissue bioprinting and maturation.

### BACKGROUND

A technique to engineer tissues is three-dimensional bioprinting. The tissues are disposed, e.g., printed, in a layer by layer approach built off of a digital computer-aided design file (e.g., CAD file). Different polymers, cells, or combinations thereof printed in this manner are additively constructed to engineer large tissues or whole organs. Initially soft protein hydrogels could not be used with the bioprinting platform due to their prevalence within the native extracellular matrix, despite being predominately used as scaffolds within the field of tissue engineering.

Conventional tissue bioprinting uses soft protein hydrogels in two-dimensional, or shallow three-dimensional layers as a support matrix for the printed tissues. These support matrixes suspend the printed tissue structures within the soft protein hydrogels, such as collagen or alginate, to be bioprinted into more complex three-dimensional structures. Since the printed tissue is secured to, and only released from the hydrogel support material after printing, new tissues or components cannot be added to the structure during the printing process.

### SUMMARY

Disclosed herein is a system and method for bioprinting of tissues in a thermally reversible support medium and delivering one or more media to the bioprinted tissues thereby supporting cellular growth and maturity over a time period that occurs during the printing process. A bioreactor is filled with the support medium, which can be composed of a mixture of betaine-modified methylcellulose and agarose microparticles. The support medium can act as a fluid (e.g., a viscosity between 1-10,000 centipoise) below a gelation temperature and a solid (e.g., a gel, e.g., a viscosity above 50,000 centipoise) above the gelation temperature.

A soft protein hydrogel, e.g., collagen or alginate, can be bioprinted into the inner volume of the bioreactor. In some embodiments, the hydrogel can include a live biologic population including one or more cell types, such as cardiomyocytes. The bioprinted hydrogel forms a three-dimensional structure within the support medium. The bioreactor and contents of the inner volume can be heated to raise the temperature of the support medium above the gelation temperature which causes the support medium to gel, thereby forming a solid support encasing the hydrogel structure. Additionally, different cells types and tissues may have different culture requirements to enable proper cell/tissue maturity. Therefore, for complex tissues to achieve proper maturity and function, different cultures may be introduced at different times according to the tissue maturation conditions.

A cover can optionally be coupled to the top surface of the bioreactor. A solution (e.g., a media) can be delivered, such as via a peristaltic pump, to an entrance port in the base of the bioreactor in fluidic connection with the bioreactor inner volume. The media can flow into the inner volume and permeate the support medium and the hydrogel structure. In some embodiments, the hydrogel structure can include a live biologic population, thus the media delivers nutrients to the live biologic population. The media can be delivered over time periods of 24 hours or more, facilitating the healthy growth and maturation of live biologic populations present in the hydrogel structure. In some embodiments, media delivered to the bioreactor flows out through fluidic connections in the cover to a fluid reservoir. In some embodiments, the media removed from the bioreactor is returned to the source reservoir, thereby recycling the media for subsequent use. Alternatively, the fluid reservoir is a waste reservoir for later disposal or used media.

When introducing additional or supplementary structures or live biologic populations to an initial structure, the support medium can be cooled below a gelation temperature thereby acting a viscous fluid. Additional hydrogel can then be bioprinted into additional structures in the support medium. The support medium is heated above the gelation temperature and media delivered to the entrance port to permeate the new structures. The support medium and structures encased therein are perfused for an additional time period. The rigidly reversible support medium can be interchangeably altered between a gel state and a solid state thereby facilitating the above process to be repeated to form complex supplementary structure as well as sustain and grow live biologics between structure printing steps.

Furthermore, a time period can be present, as desired, between repeated forming steps to allow initial structures to stabilize and/or grow over time before a new material is added. Bioprinting additional structures after delivering media allows live biologics present in previous structures to grow and mature before additional structures are printed, thereby increasing the viability of complete printed structures after completing bioprinting. In some embodiments, the support medium includes a crosslinking agent which increases the structural integrity of bioprinted structures during printing while the support medium is in a gel state, and during live biologic maturation when the support medium is in a solid state.

For example, cardiac tissue is composed of layers of anisotropically oriented tissues with a different tissue orientation with each layer. Printing each layer with a unique orientation in a step-wise fashion, allows initial layers to be matured, thereby increasing inter-biologic connection and health, before additional layers are printed. The final tissue structure can be composed of multiple healthy layers of mature cells before releasing the structure from the support medium.

In one aspect, the disclosure provides a method of forming a tissue or an organ, including disposing, in a support medium in a gel state, a composition including a live biologic; changing a state of the support medium from the gel state to a solid state; and supporting, in the support medium at the solid state, the live biologic in the composition.

In some embodiments, the support medium can be configured to reversibly change between the gel state to the solid state depending on a temperature of the support medium. The support medium, when in the gel state, can have a viscosity of no more than 10,000 centipoise, and, when in the solid state, can have a viscosity of at least 50,000 centipoise. The changing can include increasing or decreasing a temperature of the support medium across a temperature threshold, wherein the temperature threshold can be in a range of about 20° C. to about 37° C.

In some embodiments, the method can include changing the state of the support medium from the solid state to the gel state; and disposing, in the support medium in the gel state, a second composition including a live biologic, wherein the second composition can be the same or different from the composition. The supporting can occur for a time period of at least 0.1 hour, and wherein the supporting can occur after disposing of the composition, but before disposing of the second composition. The disposing can include 3D printing the composition in the support medium. The supporting can include perfusing a media to the live biologic through the support medium, wherein the perfusing includes adding the media to the support medium at a flow rate in a range from 1 cm³/min to 10 cm³/min. The supporting can include growing the live biologic.

In some embodiments, the composition can include a hydrogel, and wherein the live biologic can include live cells selected from a group consisting of cardiomyocytes, mesenchymal stem cells, induced pluripotent stem cells, cardiac progenitor cells, proepicardial cells, myocytes, hepatocytes, pneumocytes, endothelial cells, keratinocytes, nephrons, osteoblasts and any other epithelial, mesenchymal, or stem cell.

In a second aspect, the disclosure provides a bioreactor system, including a bioreactor including a housing having an outlet, and a cover that is coupleable to the housing including a liquid vent; a fluid reservoir fluidically coupled to the outlet, the liquid vent, or both, of the bioreactor; and a support medium disposed in the housing of the bioreactor; wherein the support medium is configured to reversibly change from a gel state to a solid state based on a temperature of the support medium.

In some embodiments, the support medium can include methylcellulose, agarose, betaine, transglutaminase, or combinations thereof. The support medium, when in a gel state, can have a viscosity of no more than 10,000 centipoise; and the support medium, when in the solid state, can have a viscosity of at least 50,000 centipoise.

The system can include an inlet line, an outlet line, or both, fluidically coupling the bioreactor to the fluid reservoir, and the system can include a pump fluidically coupled to the inlet line, the outlet line, or both, wherein the pump can be configured to flow a liquid media from the fluid reservoir to the bioreactor, and the pump can be configured to flow the liquid media at a flow rate of about 1 cm3/min to 10 cm3/min. The support medium can include one or more hollow channels fluidly coupled to the outlet.

In a third aspect, the disclosure provides a support medium composition, including a polymer including a temperature regulating agent; a polysaccharide particle; and a media; wherein the composition is configured to reversibly change between a gel state to a solid state depending on a temperature of the support medium.

In some embodiments, the polymer can include methylcellulose and the temperature regulating agent can include betaine, and the media can include a cell-culture media. The polysaccharide particle can include an agarose microparticle, wherein the agarose microparticle can have an average maximum particle size of about 40 μm to about 70 μm. The composition can include a crosslinking agent, wherein the crosslinking agent includes transglutaminase, 1-ethyl-3-(-3-dimethylaminopropyl) carbodiimide hydrochloride (EDC), or a combination thereof. The composition can include the polymer in a range from 2% w/v to 8% w/v and the temperature regulating agent in a range from 5% w/v to 20% w/v.

### BRIEF DESCRIPTION OF THE DRAWINGS

FIG. 1 shows a schematic representation of an exemplary bioprinting system with the bioreactor.

FIG. 2 shows a schematic representation of an exemplary bioreactor.

FIGS. 3A and 3B shows images of exemplary two stages of the bioprinting process.

FIG. 4 shows a schematic representation of an exemplary perfusion bioreactor system.

FIG. 5 shows a schematic representation of an exemplary bioreactor cover.

FIG. 6 shows a flow chart diagram of an exemplary bioprinting process.

FIG. 7 shows a series of schematic representations of exemplary method of constructing a layered structure over multiple time periods.

FIG. 8 shows a series of schematic representations of an example of constructing nested structures over a time period.

FIG. 9 shows a series of schematic representations of another example of constructing nested structures over a time period.

FIG. 10 shows a series of images depicting the visual diffusability a colored media into a support medium.

### DETAILED DESCRIPTION

The disclosure provides a system and method for bioprinting of compositions (e.g., bioinks). Various embodiments described in this document can include the bioprinting of compositions that include live biologic populations (e.g., tissues) in a thermally reversible support medium, and delivering one or more media to the bioprinted tissues thereby supporting cellular growth and maturity over a time period. This period of perfusion allows live biologic populations to grow and mature before printing additional structures into the support medium and delivering additional media to the bioprinted tissues and consecutively building higher-order structures in intervals. This method of repeating cycles of printing, and perfusing over a time period, facilitate the creation on complex tissue structures printed at different time intervals, such as layered tissues, e.g., cardiac tissues, or organs.

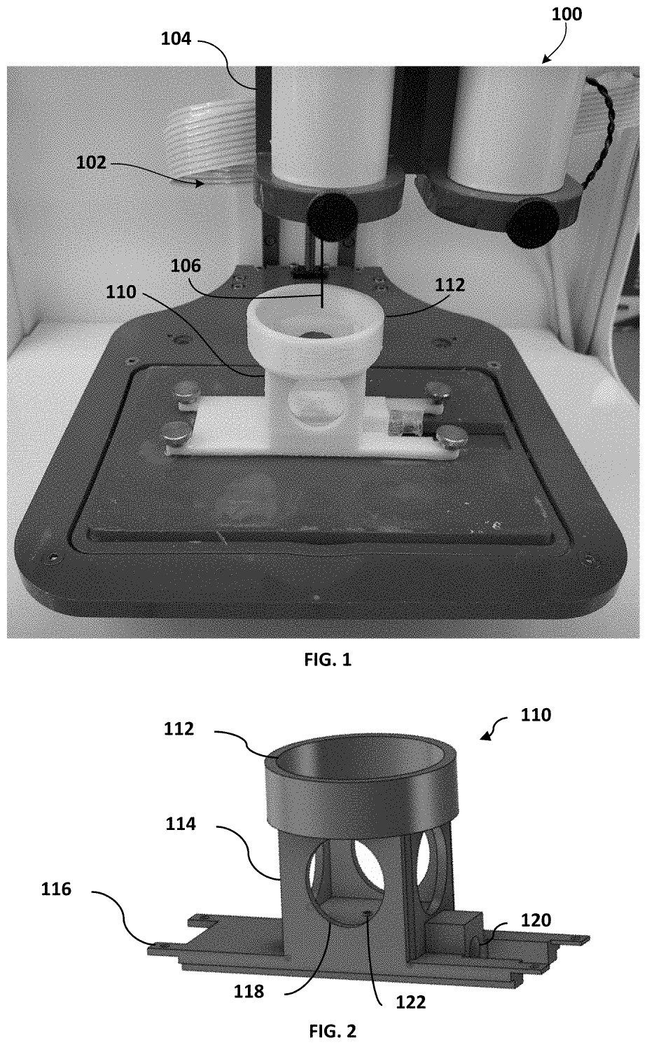

FIG. 1 shows a schematic representation of an additive manufacturing (e.g., 3D printing) bioprinting system 100 including a bioprinter 102 and a bioreactor 110. The bioprinter 102 includes a reservoir 104 containing a bioink to be disposed (e.g., printed) into the support medium. The bioink can be a hydrogel composition, such as a soft protein hydrogel, e.g., collagen or alginate. Alternative examples of bioink compositions can include polysaccharides, pluronics, PEG, gelatin methacrylate (GelMA, or PhotoCol®), fibrin, gelatin, agarose, thiolated hyaluronic acid, or decellularized extracellular matrix. In some embodiments, the bioink composition can include a population of live biologics including one or more cell types. For example, the cell types used in the bioink can include cardiomyocytes, mesenchymal stem cells, induced pluripotent stem cells, cardiac progenitor cells, proepicardial cells, myocytes, hepatocytes, pneumocytes, endothelial cells, keratinocytes, nephrons, osteoblasts and any other epithelial, mesenchymal, or stem cell. The live biologic population suspended within the bioink volume defines a cellular concentration. In some embodiments, the cellular concentration can be in a range from 10⁶ cells/mL to 10⁸ cells/mL and the cellular concentration can vary depending on the cell type, or bioink.

The bioprinter 102 holds in memory an additive manufacturing digital design file including information corresponding to a three-dimensional structure, and a process by which to construct the three-dimensional structure. A three-dimensional bioprinting software platform such as Autodesk Fusion 360 can be used to create and edit the digital design file used in the bioprinter 102. The information can include a series of spatial coordinates such as corresponding x-, y-, and z-coordinates, translation vectors to control the relative motion in the x-, y-, and z-directions between an extrusion nozzle 106 and bioreactor 110, bioink disposition rates, or other information related to the design.

The bioreactor 110 inner volume is filled with a volume of support medium in a physical state, e.g., a state of matter. The support medium composition determines a gelation temperature, above which the support medium acts as a solid, e.g., a solid state, and below which the support medium acts as a viscous fluid, e.g., a gel state. In some embodiments, the support medium has a viscosity of in a range from 50,000 centipoise and 1,000,000 centipoise (e.g., in a range from 100,000 centipoise and 1,000,000 centipoise, in a range from 300,000 centipoise and 1,000,000 centipoise, in a range from 600,000 centipoise and 1,000,000 centipoise, in a range from 900,000 centipoise and 1,000,000 centipoise, in a range from 50,000 centipoise and 900,000 centipoise, in a range from 50,000 centipoise and 600,000 centipoise, in a range from 50,000 centipoise and 300,000 centipoise, or in a range from 50,000 centipoise and 100,000 centipoise) in the solid state and a viscosity of in a range from 1 centipoise and 10,000 centipoise (e.g., in a range from 100 centipoise and 10,000 centipoise, in a range from 1000 centipoise and 10,000 centipoise, in a range from 5000 centipoise and 10,000 centipoise, in a range from 9000 centipoise and 10,000 centipoise, in a range from 1 centipoise and 9,000 centipoise, in a range from 1 centipoise and 5,000 centipoise, in a range from 1 centipoise and 1,000 centipoise, or in a range from 1 centipoise and 100 centipoise) in the gel state. In some embodiments, the support medium has a viscosity of 5,000 centipoise in the fluid phase, and a viscosity of 1,000,000 centipoise in the solid phase. In some embodiments, the support medium has a viscosity of no more than 10,000 centipoise (e.g., 10,000 or less) in the gel state and a viscosity of at least 50,000 centipoise (e.g., 50,000 centipoise or more) in the solid state.

The gelation temperature can be in a range from about 20° C. to about 37° C. (e.g., from about 24° C. to about 37° C., from about 28° C. to about 37° C., from about 32° C. to about 37° C., from about 36° C. to about 37° C., from about 20° C. to about 36° C., from about 20° C. to about 32° C., from about 20° C. to about 28° C., or from about 20° C. to 24° C.), and can depend on the on the bioink or cell type being bioprinted. In some embodiments, the gelation temperature is 35° C.

The support medium can include a colloidal suspension of agarose microparticles in a solution of betaine-modified methylcellulose. The betaine-modified methylcellulose solution is made from a 6% w/v methylcellulose solution in a 15% w/v betaine cell culture medium. Examples of cell culture medium can include maintenance medium, MEM (Minimum Essential Medium), DMEM (Dulbecco's Modified Eagle's Medium), IMDM (Iscove's Modified Dulbecco's Medium), RPMI-1640, Ham's F-10, and Ham's F-12. The 6% methylcellulose solution is blended with 1.5 parts of a 2% w/v agarose gel and 0.5 parts of cell culture medium, both the agarose and the cell culture medium also contain 15% w/v betaine. The solution is blended with a food blender resulting in agarose microparticles with a diameter in a range from 40 μm to 70 μm (e.g., 60 μm) suspended in the methylcellulose solution.

Methylcellulose is a hydrophobically modified copolymer (HMCP) including both hydrophobic and hydrophilic ends. Upon mixing with water, the hydrophilic ends hydrate and cause the formation of a viscous fluid. Raising the viscous fluid above a gelation temperature value causes the hydrophobic ends to align and create a gel network, e.g., gelation, and changing the viscous fluid to a solid state. The gelation of methylcellulose is reversible based upon whether the solution is raised above or lowered below the gelation temperature.

The gelation temperature can be modified (e.g., raised or lowered) through the addition of different temperature regulating agents within the solution which dehydrate the hydrophobic ends which thereby need less heat to align and form the gel network. For example, these temperature regulating agents include salts, such as sodium chloride or sodium bicarbonate, and sugars, such as betaine or sorbitol. Additionally and/or alternatively, electrorheological or magnetorheological fluids can form the basis for the support medium. Electrorheological, e.g., corn starch mixed in a light oil, and magnetorheological fluids, e.g., citric acid mixed in a light oil, form networks upon contact with either an electrical or magnetic field.

Transglutaminase is mixed into the support medium as a crosslinking agent which promotes protein crosslinking of the structure 302 during and after the bioprinting disposition. Transglutaminase is a biologically safe zero-length crosslinking agent and crosslinks the printed soft protein hydrogels, e.g., collagen. Other crosslinking agents can be used, depending upon the printed bioink, such as calcium chloride for alginate or thrombin for fibrinogen. Other zero-length enzymatic crosslinking agents can be used as well, such as 1-ethyl-3-(-3-dimethylaminopropyl) carbodiimide hydrochloride (EDC).

The support medium can include methylcellulose in a range from 2% and 8% (e.g., 4% and 8%, 6% and 8%, 2% and 6%, or 2% and 4%), and include betaine in a range of 5% and 20% (e.g., 10% and 20%, 15% and 20%, 5% and 15%, or 5% and 10%). For a discussion of the creation of betaine-modified methylcellulose, see Shirata et al, titled Body Heat Responsive Gelation Of Methylcellulose Formulation Containing Betaine, Bioscience, Biotechnology, and Biochemistry, 2017, Vol. 81, No. 9, which is incorporated herein by reference in its entirety.

FIG. 2 shows a schematic representation of the bioreactor 110 of FIG. 1 including a cylindrical opening 112 above a chamber housing 114 affixed to a support plate 116. The chamber housing 114 of FIG. 2 is cuboid defined by four external walls with a circular window 118 in each wall. The windows 118 are 60 mm in diameter for viewing printing or perfusion progress and composed of glass, though any transparent rigid material can perform the function. The window 118 diameter can be in a range from 10 mm to less than the width of the chamber housing 114 inner surfaces. The volume bounded by the inner surfaces of chamber housing 114 walls, chamber housing 114 floor, and opening 112 define an inner volume which contains fluids (e.g., support medium) disposed therein. The inner volume can have a volume in the range of 5 mL to 2 L (e.g., 50 mL, to 2 L, 500 mL to 2 L, 1 L to 2 L, 1.5 L to 2 L, 5 mL to 1.5 L, 5 mL to 1 L, 5 mL to 1 L, 5 mL to 500 mL, or 5 mL to 50 mL). In some embodiments, the bioreactor 110 inner volume can be greater than 2 L. The chamber housing 114 can, in general, be cuboid or cylindrical in shape.

The bioreactor 110 can be composed of any non-reactive rigid material that can be sanitized for contact with biological samples such as glass, plastic, or metal, and in some embodiments, may not include the windows 118. For example, the bioreactor 110 can be 3D printed out of polymer resin. Materials that increase thermal conductivity (e.g., metals) of the bioreactor 110 and support medium contained therein can decrease the time between the support medium being in a gel state and being in a solid state, reducing thermal diffusion and increasing the spatial resolution of printed bioink.

The bioreactor 110 includes holes at each corner of the support plate 116 for affixing to the bioprinter 102 printing platform thereby providing a stable connection. The support plate 116 shown in FIGS. 1 and 2 is rectangular (e.g., a width smaller than a length) though the support plate 116 can be designed to match the bioprinter 102 printing platform. The width and length of the support plate 116 can also be designed to match the dimensions of common bioprinting substrates, such as standard microscope slides (e.g., 25 mm width by 75 mm length) or standard well plates (e.g., 80 mm width by 125 mm length). In some embodiments, the support plate 116 can be temporarily affixed to an intermediary baseplate with different dimensions from the support plate 116 for use in a bioprinter 102 having different printing platform dimensions.

The bioreactor 110 includes a port 120 (e.g., an inlet) to which external fluid reservoirs can be connected. The port 120 can be designed to use common water-tight fluid connection mechanisms such as threaded or barbed fluid connections (e.g., Luer lock, or Luer slip connections) to provide fluidic communication between connected components such as pumps, or reservoirs and the bioreactor 110. The port 120 connects to an outlet 122 in the chamber housing 114 floor which provides fluidic communication between the port 120 and connected components, and the bioreactor 110 inner volume. The port 120 has a diameter of 6 mm, though in some embodiments can have a diameter in a range of 1 mm and 10 mm. The outlet 122 has a diameter of 1 mm, though in some embodiments can have a diameter in a range of 0.5 mm and 2.5 mm. In some embodiments, the bioreactor 110 can include more than one port 120 (e.g., two or more, e.g., four) and/or more than one outlet 122 (e.g., two or more, e.g., three, or four) and each port 120 can connect to a common outlet 122, or distinct outlets 122. In some embodiments, the port 120 is positioned vertically below the outlet 122, such as beneath the floor of the chamber housing 114. In some embodiments, the outlet 122 is positioned vertically beneath a vent in the cover of the bioreactor 110.

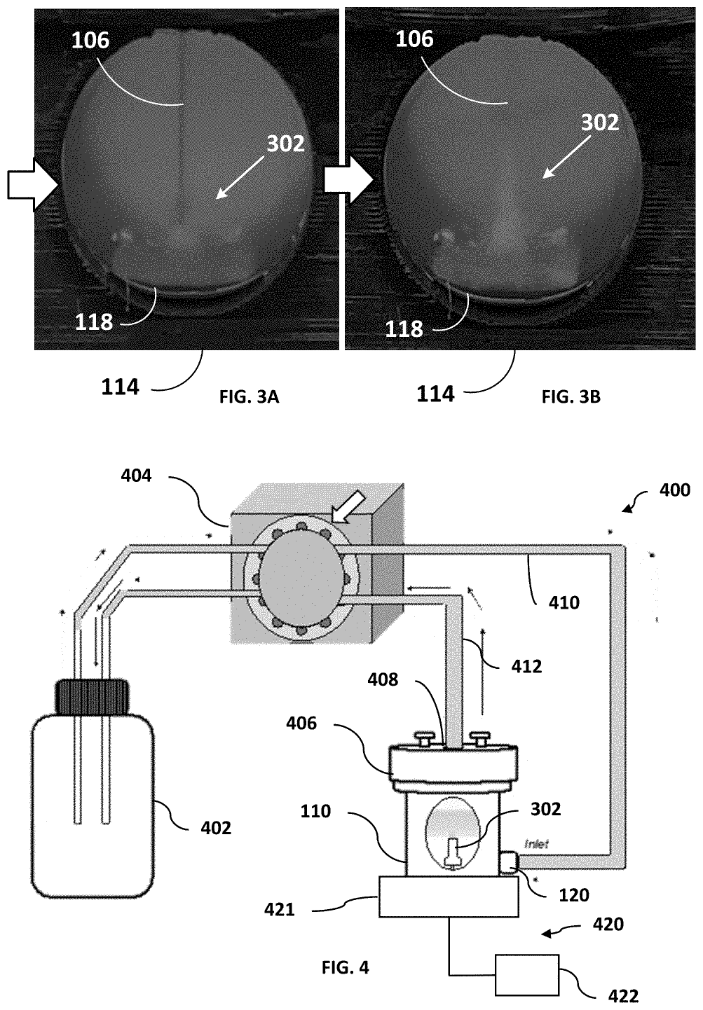

The bioprinter 102 printing method is generally an extrusion bioprinting method wherein a bioink driver mechanism, such as pneumatic gas, mechanical piston, or screw, forces the bioink through an extrusion nozzle 106 at an end of the reservoir 104, for example, the Allevi 2 Bioprinter from Allevi. FIGS. 3A-3B show example steps of a printing process as seen from a bioreactor 110 window 118. The bioprinter 102 aligns the nozzle 106 above an opening 112 in the bioreactor 110 top surface. The opening 112 provides access to the bioreactor 110 inner volume containing the support medium. The bioprinter 102 vertically repositions the nozzle 106 tip into the bioreactor 110 inner volume, and thereby into the support medium contained therein. The bioprinter 102 arrests the nozzle 106 tip at an initial spatial location (e.g., x, y, z coordinates) determined by the digital design file loaded in the bioprinter 102.

While maintaining the nozzle 106 tip within the support medium, the bioprinter 102 disposes bioink at a flow rate while creating relative motion between the nozzle 106 and the bioreactor 110. The support medium displaced by the nozzle 106 relative motion flows around the nozzle 106 and maintains disposed bioink at the disposed spatial location. The flow rate, and nozzle 106 relative velocity can depend on the bioink parameters, temperature and/or viscosity of the support medium. In some examples, the flow rate can be in a range from 0.1 cm³/min to 0.5 cm³/min and nozzle relative velocity can be in a range from 1 mm/s to 20 mm/s. The nozzle 106 is shown in FIG. 3A as vertically aligned within the support medium and having extruded a portion of bioink into a three-dimensional structure 302. FIG. 3B depicts a later time point at which the structure 302 has been additively manufactured to include additional bioink in a vertically-aligned formation.

The bioprinter 102 performs the printing process determined by the digital design file loaded in memory until completion and removes the nozzle 106 from the support medium. The temperature of the support medium is raised above the gelation temperature, thereby causing the support medium to act as a solid. In some embodiments, the temperature of the support medium is raised above the gelation temperature by placing the printed object in a biological incubator held at a temperature above the gelation temperature of the support medium. The spatial orientation and position of printed bioink contained therein is maintained by the gelled support medium.

A schematic representation of perfusion bioreactor system 400 is shown in FIG. 4. The example perfusion bioreactor system 400 includes a fluid reservoir 402 configured to receive a liquid media and a pressure source 404 in fluid connection with a cover 406 coupled to the opening 112 of the bioreactor 110 containing the support medium and printed structure 302. A feed line 410 (e.g., an inlet line) and a return line 412 (e.g., outlet line) are shown creating a fluid circuit between the fluid reservoir 402, the pressure source 404, and the bioreactor 110 inner volume. The fluid contained in the fluid reservoir 402 can be a media (e.g., a growth media) including one or more nutrients, growth factors, antibiotics, antivirals, antimicrobials, antifungals, buffers, and/or salts for preserving the structure 302 and supporting any live biologic populations contained therein through growth and replication cycles (e.g., growing). In some embodiments, the fluid contained in the fluid reservoir 402 can be an oxygen carrier fluid, such as blood, or hemoglobin-based oxygen carrier (HBOC).

The pressure source 404 is a pump (e.g., a peristaltic pump) is configured to flow a fluid (e.g., liquid media) from the fluid reservoir 402 to the example bioreactor 110 by providing positive pressure to the port 120 via feed line 410 and negative pressure to a liquid vent 408 extending through the cover 406. In some embodiments, the pressure source 404 can be alternative pumps capable of creating pressure differentials and driving fluids at low flow rates such as a diaphragm pump, centrifugal pump, or piston pump.

FIG. 4 illustrates a temperature controlling apparatus 420 in contact with the bioreactor 110, including a heating/cooling element 421 and a temperature controller 422. The heating/cooling element 421 is operable to raise and/or lower the temperature of the bioreactor 110 and support medium contained in the chamber housing 114 across the gelation temperature. For example, the heating/cooling element 421 can be an electric heating/cooling element 421 such as a Peltier heating/cooling element 421, or a fluid-based heat-exchanger. In some embodiments, the heating/cooling element 421 is integrated with the bioreactor 110, for example, integrated with the chamber housing 114. The temperature controller 422 includes the electronics, user interfaces, and control software to operate the heating/cooling element 421 to cause the temperature of the bioreactor 110 to raise and/or lower to a temperature value set by the user or by the bioprinter 102.

The perfusion bioreactor system 400 of FIG. 4 includes a fluidic circuit wherein the pressure source 404 draws fluid (e.g., a media) from the fluid reservoir 402 and dispenses drawn fluid into the port 120 through the feed line 410.

The fluid entering the port 120 exits outlet 122. The support medium can include one or more hollow channels. In some embodiments, the hollow channel is cylindrical void in the support medium connecting two surfaces of the support medium, such as two opposing surfaces, e.g., the top and bottom surfaces. An opening of a hollow channel can be arranged above an opening 122 such that fluid exiting outlet 122 perfuses through the support medium and structure 302 including any live biologics supported therein. A continual flow of fluid into the port 120, exits outlet 122, displaces fluid present in the bioreactor 110 inner volume and in the hollow channel, which exits through a liquid vent 408. The pressure source 404 creates a negative pressure within the return line 412, drawing displaced fluid into the pressure source 404 and driving it to the fluid reservoir 402. In embodiments in which bioreactor 110 includes more than one outlet 122, the number of hollow channels can be equal to or greater than the number of outlets 122.

In some embodiments, the fluidic circuit of the perfusion bioreactor system 400 can include additional feed lines 410 and return lines 412 coupling additional perfusion bioreactor system 400 components such as more than one bioreactors 110, more than one pressure source 404, and or more than one fluid reservoir 402.

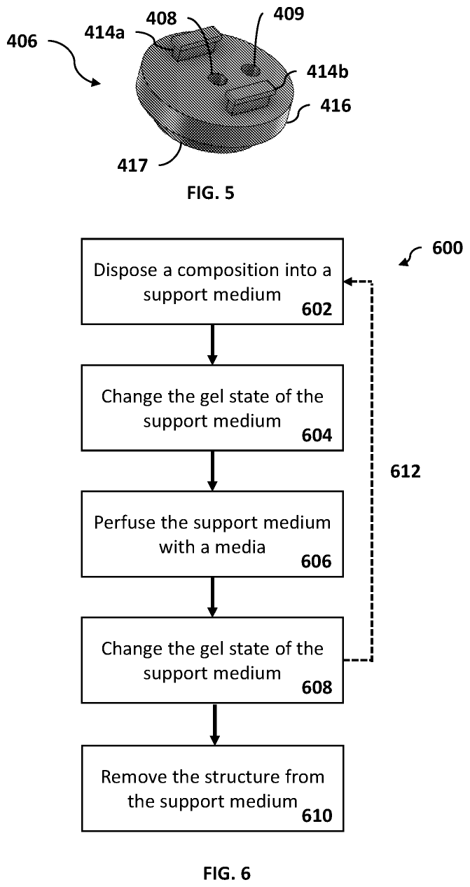

FIG. 5 shows a schematic representation of the cover 406 including a view of the liquid vent 408. The cover 406 seals the opening 112 against liquid and gas ingress/egress from the opening 112 circumference and can prevent potential introduction of foreign microbes to maintain a sterile environment within the chamber housing 114. The liquid vent 408 connects the top surface of the cover 406 with the lower surface facing the chamber housing 114 chamber housing 114 inner volume. The liquid vent 408 can be designed to include any water-tight fluid connection described herein, such as the port 120 connections described above. In some embodiments, the cover 406 includes an air vent 409 which can be used for circulating air, e.g., filtered aid, through the inner volume, pressure equilibration, or venting of gasses (e.g., bubbles). The cover 406 includes two optional posts 414a and 414b providing static points for applying rotational torque when attaching or removing the cover 406 from the bioreactor 110.

The cover 406 is composed of two cylindrical portions including respective diameters connected by a planar surface. The lower portion 417 outer diameter is less than the inner diameter of the opening 112 and when coupled to the opening 112 creates a pressure- and fluid-tight seal (e.g., hermetically, and/or fluidically sealed) with the opening 112 when the cover 406 is coupled. In some embodiments, the lower portion 417 and opening 112 include threading to mate the cover 406 to the opening 112 via a screwing motion of the cover 406. The upper portion 416 outer diameter is greater than the lower cylinder diameter such that the planar surface connecting the upper portion and the lower portion and the opening 112 upper surface are flush when the cover 406 is coupled to the bioreactor 110. In some embodiments, additional sealing components, such as gaskets, or O-rings, can be included on, or embedded in, the lower cylinder or planar surface to improve the fluid sealing capability of the cover 406.

Referring again to FIG. 4, the pressure source 404, feed line 410 and return line 412 inner diameter, port 120 diameter, and liquid vent 408 diameter can be adjusted to control the fluid flow rate through the port 120 and liquid vent 408. The flow rate can be 1 cm³/min or more, for example, in a range from 1 cm³/min to 10 cm³/min. Fluid can be circulated through the fluidic circuit for a time period over which the fluid flows around and through (e.g., perfuses) inner volume, structure 302, and live biologic populations contained therein. In some embodiments, the inner volume and structure 302 can be perfused in a range between 0.1 hours and 72 hours (e.g., 1 hours and 72 hours, 5 hours and 72 hours, 10 hours and 72 hours, 20 hours and 72 hours, 24 hours and 72 hours, 36 hours and 72 hours, 48 hours and 72 hours, 0.1 hours and 48 hours, 0.1 hours and 36 hours, 0.1 hours and 24 hours, 0.1 hours and 20 hours, 0.1 hours and 10 hours, 0.1 hours and 5 hours, or 0.1 hours and 1 hours).

Additional structures 302 can be added to the support medium by reducing the support medium temperature below the gelation temperature and detaching the cover 406 from the bioreactor 110. The bioreactor 110 is reaffixed to the bioprinter 102, and additional bioink bioprinted into the support medium according to additional instructions within the first digital design file, or instructions within a second digital design file to form a second structure. The nozzle 106 is removed from the support medium and the support medium temperature raised above the gelation temperature to maintain the first and second structures 302. The first and second structures 302 can then be perfused according to the first or a second time period.

FIG. 6 is a flow chart showing the steps of the bioprinting process 600. A bioink is disposed into a support medium (step 602) contained within the inner volume of a bioreactor 110. The support medium is a thermally reversible gel having a gelation temperature above which the support medium is in a solid state, e.g., acts as a solid, and below which the support medium is in a gel state, e.g., acts as a viscous fluid. The nozzle 106 is positioned through an opening 112 of the bioreactor 110 within the support medium in the gel state and the bioprinter 102 disposes a bioink into the support medium. The bioprinter 102 creates relative motion between the nozzle 106 and the bioreactor 110 and disposes additional bioink to form a structure 302 maintained by the support medium. The bioreactor 110 withdraws the nozzle 106 from the support medium. A cover 406 is coupled to the opening 112 sealing the support medium into the chamber housing 114 chamber housing 114 inner volume.

The support medium state temperature is raised above the gelation temperature to change the support medium state (step 604) to act as a solid, e.g., the solid state. The solid state of the support medium maintains the spatial position and resolution of the printed structure 302.

A fluid is delivered to support medium contained in the bioreactor 110 inner volume (step 606). A pressure source 404 delivers the fluid from a fluid reservoir 402 to the port 120 which perfuses the support medium and structure 302 maintained therein. Excess fluid exits through a liquid vent 408 in the cover 406 and is drawn through a return line 412 to the pressure source 404 and delivered to the fluid reservoir 402 creating a fluidic circuit between the chamber housing 114 chamber housing 114 inner volume and the fluid reservoir 402. The pressure source 404 circulates the fluid through the inner volume and fluid reservoir 402 for a time period as the structure 302 and live biologic populations mature and multiply.

The support medium temperature can be lowered below the gelation temperature to change the support medium state (step 608) to act as a fluid. The cover 406 removed and the structure 302 can then be removed from the support medium. Alternatively, steps 602, 604, 606, and 608 can be repeated (step 612) at least one more time to dispose (602) at least one more structure 302 to the support medium, replace the cover 406, change (604) the support medium state to a solid state, perfuse (606) structures 302 within the support medium, and change (608) the support medium state to a viscous fluid, e.g., a gel state.

The following examples are provided to further elucidate the advantages and features of the present application, but are not intended to limit the scope of the application. The examples are for the illustrative purposes only.

### EXAMPLES

### Example 1: Bioprinting Three Layered Structures

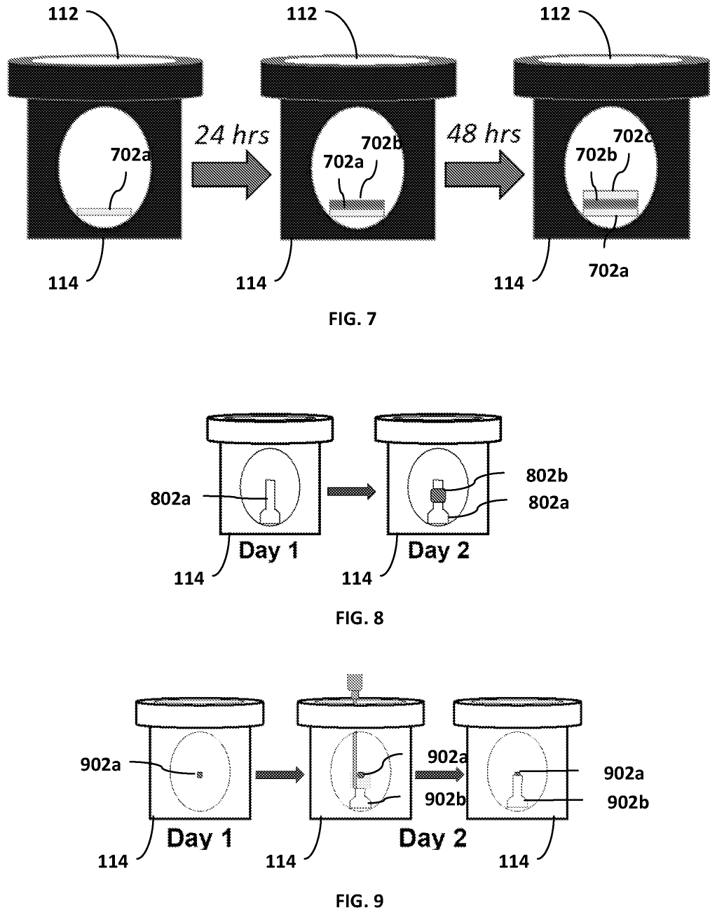

FIG. 7 shows an example bioreactor 110 chamber housing 114 chamber housing 114 with the opening 112 oriented upward. A first structure 702a composed of a collagen hydrogel bioink is shown printed above the floor of the chamber housing 114 chamber housing 114 and suspended in the support matrix. The first structure 702a is a flat square-shaped structure with a 1 mm thickness and 1 cm lateral dimensions. The support matrix temperature is raised above the gelation temperature to act as a solid and a fluid is perfused through the support matrix by the perfusion bioreactor system 400 (not shown) for a first time period, e.g., a 24 hour time period as shown in FIG. 7.

The support matrix temperature is lowered below the gelation temperature such that the matrix acts as a fluid. A second structure 702b is printed with collagen hydrogel bioink including a blue dye is printed extending above the top surface of the first structure 702a. The support matrix temperature is raised above the gelation temperature to act as a solid and a fluid is perfused through the support matrix by the perfusion bioreactor system 400 (not shown) for a second time period, e.g., a second 24 hour time period for a total of 48 hours after the first structure 702a was bioprinted.

The support matrix temperature is lowered below the gelation temperature such that the matrix acts as a fluid. A third structure 702c collagen hydrogel bioink is printed extending above the top surface of the second structure 702b, thereby creating a three-layer structure from collagen hydrogel bioinks within the bioreactor 110.

### Example 2: Secondary Structures Surrounding Primary Structures

An example of printing secondary structures surrounding primary structures is shown in FIG. 8. In this example, a less complex secondary structure is printed surrounding a more complex primary structure already supported by the support matrix. FIG. 8 shows an example bioreactor 110 chamber housing 114 chamber housing 114 with the opening 112 oriented upward. A first structure 802a composed of a collagen hydrogel bioink is shown printed above the floor of the chamber housing 114 chamber housing 114 and suspended in the support matrix. The first structure 802a is a vertically oriented body including a lower first cylindrical portion and an upper second cylindrical portion, the first cylindrical portion having a wider diameter than the second cylindrical portion. The support matrix temperature is raised above the gelation temperature to act as a solid and a fluid is perfused through the support matrix by the perfusion bioreactor system 400 (not shown) for a first time period.

The support matrix temperature is lowered below the gelation temperature such that the matrix acts as a fluid. A cylindrical second structure 802b is printed with collagen hydrogel bioink including a blue dye surrounding the upper second cylindrical portion of the first structure 802a. The inner diameter of the second structure 802b is larger than the outer diameter of the upper second cylindrical portion of the first structure 802a. The support matrix temperature is raised above the gelation temperature to act as a solid supporting the second structure 802b surrounding a portion of the first structure 802a.

An example of printing primary structures around secondary structures is shown in FIG. 9. In this example, a more complex primary structure is printed surrounding a less complex secondary structure already supported by the support matrix. FIG. 9 shows an example chamber housing 114 chamber housing 114 with the opening 112 oriented upward and a first structure 902a composed of a collagen hydrogel bioink including a blue dye is shown printed above the floor of the chamber housing 114 chamber housing 114 and suspended in the support matrix. The support matrix temperature is raised above the gelation temperature to act as a solid and a fluid is perfused through the support matrix by the perfusion bioreactor system 400 (not shown) for a first time period.

The support matrix temperature is lowered below the gelation temperature such that the matrix acts as a fluid. A cylindrical second structure 902b is printed with collagen hydrogel bioink similar to the first structure 802a of FIG. 8. The upper second cylindrical portion of the second structure 902b surrounds the outer diameter of the first structure 902a. The support matrix temperature is raised above the gelation temperature to act as a solid supporting the second structure 802b surrounding a portion of the first structure 802a.

### Example 3: Fluid Diffusability of the Support Medium

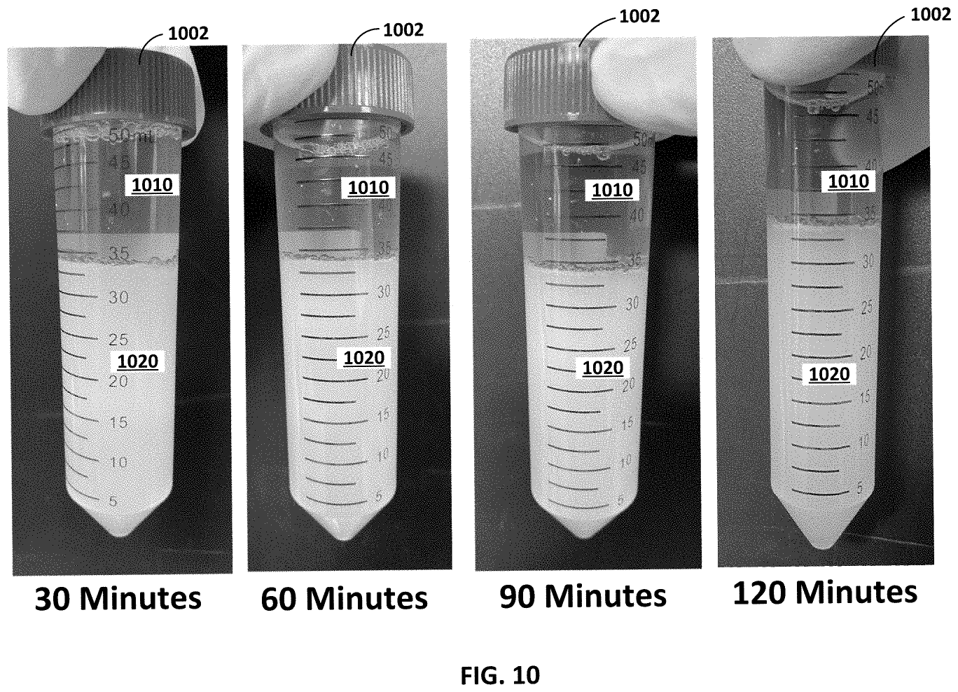

FIG. 10 is a series of images showing the fluid (e.g., media 1010) diffusability of the support medium 1020. The support medium 1020 is composed of 6% w/v methylcellulose, 2% w/v agarose microparticles, 15% w/v betaine, and cell culture media. The images of FIG. 10 depict a conical tube 1002 including a support medium 1020 volume beneath a media 1010 volume and an arrow denoting the interface between the two materials. The media 1010 includes a colored dye such that media 1010 diffusing into the support medium 1020 shows a visual color change in the upper portion of the support medium 1020. The images, from left to right, depict the diffusion interface after the time periods denoted beneath the respective images, after 30 minutes, after 60 minutes, after 90 minutes, and after 120 minutes, respectively. No visually apparent diffusion of the media 2010 including a colored dye into the support medium 1020 was recorded.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of forming a tissue or an organ, comprising:
disposing, in a support medium in a gel state, a composition comprising a live biologic;
changing a state of the support medium from the gel state to a solid state; and
supporting, in the support medium at the solid state, the live biologic in the composition.

2. The method of claim 1, wherein the support medium is configured to reversibly change between the gel state to the solid state depending on a temperature of the support medium.

3. The method of claim 1, wherein the support medium, when in the gel state, has a viscosity of no more than 10,000 centipoise, and, when in the solid state, has a viscosity of at least 50,000 centipoise.

4. The method of claim 1, wherein the changing comprises increasing or decreasing a temperature of the support medium across a temperature threshold, wherein the temperature threshold is in a range of about 20° C. to about 37° C.

5. The method of claim 1, wherein the method comprises:
changing the state of the support medium from the solid state to the gel state; and
disposing, in the support medium in the gel state, a second composition comprising a live biologic, wherein the second composition is the same or different from the composition.

6. The method of claim 5, wherein the supporting occurs for a time period of at least 0.1 hour, and wherein the supporting occurs after disposing of the composition, but before disposing of the second composition.

7. The method of claim 1, wherein the disposing comprises 3D printing the composition in the support medium.

8. The method of claim 1, wherein the supporting comprise perfusing a media to the live biologic through the support medium, wherein the perfusing comprises adding the media to the support medium at a flow rate in a range from 1 cm³/min to 10 cm³/min.

9. The method of claim 1, wherein the supporting comprises growing the live biologic.

10. The method of claim 1, wherein the composition comprises a hydrogel, and wherein the live biologic comprises live cells selected from a group consisting of cardiomyocytes, mesenchymal stem cells, induced pluripotent stem cells, cardiac progenitor cells, proepicardial cells, myocytes, hepatocytes, pneumocytes, endothelial cells, keratinocytes, nephrons, osteoblasts and any other epithelial, mesenchymal, or stem cell.

11. A bioreactor system, comprising:
a bioreactor comprising a housing having an outlet, and a cover that is coupleable to the housing comprising a liquid vent;
a fluid reservoir fluidically coupled to the outlet, the liquid vent, or both, of the bioreactor; and
a support medium disposed in the housing of the bioreactor;
wherein the support medium is configured to reversibly change from a gel state to a solid state based on a temperature of the support medium.

12. The system of claim 11, wherein the support medium comprises methylcellulose, agarose, betaine, transglutaminase, or combinations thereof.

13. The system of claim 11,
wherein the support medium, when in a gel state, has a viscosity of no more than 10,000 centipoise; and
wherein the support medium, when in the solid state, has a viscosity of at least 50,000 centipoise.

14. The system of claim 11, wherein the system comprises an inlet line, an outlet line, or both, fluidically coupling the bioreactor to the fluid reservoir,
wherein the system comprises a pump fluidically coupled to the inlet line, the outlet line, or both, wherein the pump is configured to flow a liquid media from the fluid reservoir to the bioreactor, and wherein the pump is configured to flow the liquid media at a flow rate of about 1 cm³/min to 10 cm³/min.

15. The system of claim 11, wherein the support medium includes one or more hollow channels fluidly coupled to the outlet.

16. A support medium composition, comprising:
a polymer comprising a temperature regulating agent;
a polysaccharide particle; and
a media;
wherein the composition is configured to reversibly change between a gel state to a solid state depending on a temperature of the support medium.

17. The composition of claim 16, wherein the polymer comprises methylcellulose and the temperature regulating agent comprises betaine, and the media comprises a cell-culture media.

18. The composition of claim 16, wherein the polysaccharide particle comprises an agarose microparticle, wherein the agarose microparticle has an average maximum particle size of about 40 μm to about 70 μm.

19. The composition of claim 16, wherein the composition comprises a crosslinking agent, wherein the crosslinking agent comprises transglutaminase, 1-ethyl-3-(-3-dimethylaminopropyl) carbodiimide hydrochloride (EDC), or a combination thereof.

20. The composition of claim 16, wherein the composition comprises the polymer in a range from 2% w/v to 8% w/v and the temperature regulating agent in a range from 5% w/v to 20% w/v.
